(19) United States
(12) Patent Application Publication
(10) Pub. No.: US 2014/0343319 A1
(43) Pub. Date: Nov. 20, 2014

(54) PROCESS FOR PREPARING ACRYLIC ACID WITH HIGH SPACE-TIME YIELD

(71) Applicant: BASF SE, Ludwigshafen (DE)

(72) Inventors: Michael GOEBEL, Mannheim (DE); Christian Walsdorff, Ludwigshafen (DE); Marco Hartmann, Woerth (DE); Nicolai Tonio Woerz, Darmstadt (DE); Tim Blaschke, Stuttgart (DE); Philipp Gruene, Mannheim (DE)

(73) Assignee: BASF SE, Ludwigshafen (DE)

(21) Appl. No.: 14/277,414

(22) Filed: May 14, 2014

Related U.S. Application Data
(60) Provisional application No. 61/822,950, filed on May 14, 2013.

(30) Foreign Application Priority Data
May 14, 2013 (DE) 10 2013 008 207.2

(51) Int. Cl. C07C 51/377

(52) U.S. Cl. CPC C07C 51/377
USPC 562/599

(57) ABSTRACT

In a process for preparing acrylic acid, a reaction gas which comprises a gaseous formaldehyde source and gaseous acetic acid and in which the partial pressure of the formaldehyde source, calculated as formaldehyde equivalents, is at least 85 mbar and in which the molar ratio of the acetic acid to the formaldehyde source, calculated as formaldehyde equivalents, is at least 1 is contacted with a solid condensation catalyst. The space-time yield can be enhanced significantly by increasing the partial pressure of the reactants. The space-time yield remains high even after prolonged process duration.

The present invention relates to a process for preparing acrylic acid by reaction of formaldehyde with acetic acid.

At present, acrylic acid is prepared on the industrial scale essentially by heterogeneously catalyzed two-stage partial oxidation of propene (see, for example, DE-A 103 36 386).

One advantage of this procedure is that it has a comparatively high target product selectivity based on propene converted.

At present, propene is prepared on the industrial scale essentially proceeding from mineral oil or propane-containing natural gas. In view of the foreseeable scarcity of the fossil resources of mineral oil and natural gas, however, there will be a future need for processes for preparing acrylic acid from alternative and/or renewable raw materials.

The preparation of acrylic acid from acetic acid and formaldehyde is prior art. One reason why this procedure is advantageous is that formaldehyde is obtainable by partial oxidation of methanol. Methanol can be produced via synthesis gas (gas mixtures composed of carbon monoxide and molecular hydrogen) in principle from all carbonaceous fossil base materials and all carbonaceous renewable raw materials.

US 2012/0071688 A1 discloses a process for preparing acrylic acid from methanol and acetic acid, which comprises a plurality of process steps. In one process step, methanol is reacted with molecular oxygen to give formaldehyde. In a further process step, the formaldehyde is reacted with acetic acid to give acrylic acid, the acrylic acid at first being obtained as part of a product gas mixture. The product gas mixture is divided in a further process step into at least three streams, one of the streams consisting predominantly of acetic acid and being recycled as the reactant into the process, and another stream consisting predominantly of acrylic acid.

WO 2013/028356 discloses a process for preparing unsaturated acids, such as acrylic acids or the esters thereof (alkyl acrylates), wherein an alkylcarboxylic acid is contacted with an alkylenating agent, such as a methylenating agent, under conditions suitable for preparation of the unsaturated acid or the acrylates. The alkylcarboxylic acid is used in a molar excess in some embodiments and in a molar deficiency in other embodiments relative to the alkylenating agent. In the examples, a gas comprising 9.1% acetic acid and 17.3% formaldehyde is converted over titanium pyrophosphate catalysts, achieving space-time yields of up to 57.3 g of acrylates (acrylic acid and methacrylate) per liter of catalyst used and hour at 370° C.

European patent EP 0 958 272 discloses a process for preparing an α,β-unsaturated carboxylic acid, which comprises contacting formaldehyde or a source of formaldehyde with a carboxylic acid or a carboxylic anhydride comprising an oxide of niobium. The examples describe the reaction of 15.5 mmol of formaldehyde per hour with 72.2 mmol of propanoic acid per hour in the presence of 220 mmol of nitrogen per hour at a pressure of 3 bar.

U.S. Pat. No. 4,165,438 discloses a process for preparing acrylic acid and esters thereof, wherein the co-reactants, formaldehyde and a lower alkylcarboxylic acid or the lower alkyl esters thereof, are converted in the gas phase at about 300° C. to 500° C. in the presence of a catalyst. The catalyst consists principally of vanadium orthophosphate having an intrinsic surface area of about 10 to about 50 m²/g and a P/V atomic ratio of 1:1 to 1.5:1. In the examples, a reaction mixture consisting of acetic acid, formaldehyde and water (molar ratio 10:1:2.8) is converted.

Journal of Catalysis, 1987, 107, 201-208 describes experiments in which the formation of acrylic acid from formaldehyde and acetic acid has been examined over a V₂O₅—P₂O₅ catalyst as a function of the reactant concentrations present in the gas stream at 325° C. under atmospheric pressure. It has been found that the formation of acrylic acid in a gas stream comprising 7.26 mol % of acetic acid rises only up to a concentration of 2 mol % of formaldehyde. The yield of acrylic acid based on the amount of formaldehyde used, in a gas stream which comprised 2.85 mol % of formaldehyde, rose only up to a concentration of 7 mol % of acetic acid.

Similar experiments in which propanoic acid has been used in place of acetic acid are described in Journal of Catalysis, 1988, 36, 221-230. In a gas stream which comprised 1.0 or 2.5 mol % of formaldehyde, the formation of methacrylic acid at 260° C. rose only up to a propanoic acid concentration of about 5 mol %. The yield of methacrylic acid based on the amount of formaldehyde used, in a gas stream which comprised 6.5 mol % of propanoic acid, rose only up to a concentration of 2 mol % of formaldehyde.

The catalysts are gradually deactivated in the reaction of the alkanecarboxylic acids with formaldehyde. It is assumed (see, for example, J. A. Moulijn, Applied Catalysis A 2001, 212, 3-16) that the formation of carbonaceous deposits plays an important role in the deactivation of catalysts. The formation of carbonaceous deposits depends on the conditions, such as the temperature and the partial pressure of the reactants and products. It is caused by reactions such as the unwanted polymerization and dehydrogenation of organic reactants or products. These reactions lead to the formation of a highly carbonaceous material on the surface of the catalyst, which makes the active site inaccessible and hence deactivates the catalyst. A high concentration of organic reaction gas constituents generally promotes the formation of carbonaceous deposits.

Additional disadvantages of the prior art are the excessively low space-time yield of acrylic acid, and the significant decrease in space-time yield of these condensation products with increasing process duration.

It was an object of the present invention to provide a process for preparing acrylic acid which does not have the disadvantages of the prior art processes. More particularly, it was an object of the present invention to provide a process which ensures a high space-time yield of the process product. It was also an object of the present invention to configure the process such that a high space-time yield is still achieved even after prolonged process duration.

It has been found that the space-time yield can be increased significantly by increasing the partial pressure of the reactants. This is surprising particularly in view of the above-described prior art (Journal of Catalysis, 1987, 107, 201-208, Journal of Catalysis, 1988, 36, 221-230).

It has also been found that, at the partial pressure of the reactants which is high in accordance with the invention, the space-time yield remains high even after prolonged process duration, i.e. the decrease in the space-time yield is suppressed.

The object is achieved by a process for preparing acrylic acid, wherein a reaction gas which comprises a gaseous formaldehyde source and gaseous acetic acid and in which the partial pressure of the formaldehyde source, calculated as formaldehyde equivalents, is at least 85 mbar and in which the molar ratio of the acetic acid to the formaldehyde source, calculated as formaldehyde equivalents, is at least 1 is contacted with a solid condensation catalyst in order to obtain a product gas comprising acrylic acid.

All pressure figures in this document relate to absolute pressures.

The partial pressure of the formaldehyde source, calculated as formaldehyde equivalents, in the reaction gas is preferably at least 100 mbar, more preferably at least 120 mbar and most preferably at least 135 mbar. The expression “calculated as formaldehyde equivalents” refers to the actual or theoretical state in which the theoretical maximum number of formaldehyde molecules is released from the formaldehyde source. For example, the percentage by volume of trioxane in the reaction gas is multiplied by 3 and multiplied by the total pressure of the reaction gas in order to obtain the partial pressure calculated as formaldehyde equivalents.

In a preferred embodiment, the ratio of the partial pressure of the formaldehyde source, calculated as formaldehyde equivalents, to the total pressure of the reaction gas is 0.1 to 0.5, preferably 0.1 to 0.3, more preferably 0.11 to 0.2 and most preferably 0.12 to 0.17.

In a preferred embodiment, the ratio of the partial pressure of acetic acid to the total pressure of the reaction gas is 0.5 to 0.9, preferably 0.6 to 0.85.

The reaction gas may comprise at least one inert diluent gas, especially an inert diluent gas other than steam. The ratio of the partial pressure of the inert diluent gas to the total pressure of the reaction gas may be up to 0.5, preferably up to 0.4 and more preferably up to 0.3. An inert diluent gas is understood to mean a gas which behaves inertly under the conditions which exist in the process according to the invention. Any individual inert reaction gas constituent in the process according to the invention is preserved chemically unchanged to an extent of more than 95 mol %, preferably to an extent of more than 97 mol %, or to an extent of more than 98 mol %, or to an extent of more than 99 mol %. Examples of inert diluent gases are N₂, CO₂, H₂O and noble gases such as Ar, and mixtures of the aforementioned gases. The inert diluent gas used is preferably molecular nitrogen. Suitably, 60 to 100% by volume, preferably 80 to 100% by volume and more preferably at least 90 to 100% by volume of the inert diluent gas other than steam is accounted for by molecular nitrogen.

In particular embodiments, the reaction gas does not comprise any inert diluent gas other than steam.

In the reaction between formaldehyde and acetic acid to give acrylic acid, water is released (condensation reaction). Steam assumes a special role as an inert diluent gas. It is obtained as a by-product of the condensation reaction. Water is also present in some of the formaldehyde sources mentioned below and may be introduced into the process therewith as steam. Water may also be present as an impurity in acetic acid and, after the vaporization of the acetic acid, may be introduced into the process in the form of steam. Steam generally impairs the desired condensation reaction. The ratio of the partial pressure of steam to the total pressure of the reaction gas is preferably 0 to 0.2, more preferably 0 to 0.15 and especially preferably 0 to 0.1.

The reaction gas may comprise at least one reaction gas constituent which is predominantly solid under standard conditions (20° C., 1013 mbar), in the form of a “solid reaction gas constituent” (for example some of the formaldehyde sources described below, such as trioxane). The reaction gas may also comprise at least one reaction gas constituent which is predominantly liquid under standard conditions, as a “liquid reaction gas constituent”. The reaction gas may also comprise a reaction gas constituent which is predominantly gaseous under standard conditions, as a “gaseous reaction gas constituent” (e.g. formaldehyde).

The production of the reaction gas may comprise the conversion of nongaseous reaction gas constituents to the gas phase and the combination of all the reaction gas constituents. The conversion to the gas phase and the combination can be effected in any desired sequence. At least one of the gaseous reaction gas constituents and/or a solid reaction gas constituent may also first be absorbed at least partly in at least one liquid reaction gas constituent and then be converted to the gas phase together with the liquid reaction gas constituent.

The conversion to the gas phase is effected by vaporization, preferably by supplying heat and/or reducing the pressure. The nongaseous reaction gas constituents can be introduced into gaseous reaction gas constituents in order to promote the vaporization of the nongaseous reaction gas constituents. Preference is given to initially charging a solution which comprises at least one liquid reaction gas constituent and may comprise other reaction gas constituents in a reservoir vessel, and conveying the initially charged solution, for example with the aid of a pump, at the desired volume flow rate into a gaseous stream of preheated reaction gas constituents. The initially charged solution can be combined with the gaseous stream of preheated reaction gas constituents, for example, in a vaporizer coil.

In the production of the reaction gas, it should particularly be ensured that some of the formaldehyde sources mentioned below are in liquid, solid and/or gaseous form under standard conditions. According to the choice of formaldehyde source, the formaldehyde can be released from the formaldehyde source before and/or after the conversion to the gaseous phase.

The catalyst may take the form of a fluidized bed. The catalyst preferably takes the form of a fixed bed.

Preferably, the catalyst is disposed in a reaction zone. The reaction zone may be disposed in a heat exchanger reactor having at least one primary space and at least one secondary space. The primary space and the secondary space are separated from one another by a dividing wall. The primary space comprises the reaction zone in which at least the catalyst is disposed. A fluid heat carrier flows through the secondary space. Heat is exchanged through the dividing wall with the purpose of monitoring and controlling the temperature of the reaction gas in contact with the catalyst (of heating the reaction zone).

In addition, the reaction zone may be disposed in an adiabatic reactor. In an adiabatic reactor, the heat of reaction is not removed via a dividing wall by thermal contact with a heat carrier, for instance a fluid heat carrier, but remains predominantly in the reaction zone. As a result of the adiabaticity, the temperature of the reaction gas or product gas in an exothermic reaction increases over the reactor length.

The reaction gas is generally contacted with the catalyst at a reaction temperature of 250 to 400° C., preferably at 260 to 390° C., more preferably at 270 to 380° C., especially preferably at 290 to 370° C., more especially preferably at 290 to 340° C., very especially preferably at 300 to 325° C. and even more especially preferably at 302 to 322° C. The reaction temperature is the temperature of the reaction gas within the catalyst bed averaged over the volume of the catalyst. The reaction temperature can be calculated from the temperature profile of the catalyst bed. In an isothermal reaction, the reaction temperature corresponds to the temperature which is established at the outer reactor wall. The temperature can be set using a heater. Preference is given to supplying the reaction gas to the reaction zone already with a temperature in the range from 160 to 400° C. The reaction gas can be contacted with solid inert material before contacting it with the catalyst. In contact with the solid inert material, the temperature of the reaction gas can be set to the value with which the reaction gas is to come into contact with the catalyst.

The total pressure of the reaction gas, i.e. the pressure over the catalyst which exists in the reaction gas, may be either greater than or equal to 1 bar or less than 1 bar. The total pressure of the reaction gas is preferably 1.0 bar to 50 bar, more preferably 1.0 bar to 20 bar, especially preferably 1.0 bar to 10 bar and most preferably 1.0 bar to 6.0 bar.

The condensation catalyst is preferably selected from
(i) catalysts having an active composition which comprises a multielement oxide and comprises at least one first element selected from titanium, vanadium, chromium, iron, cobalt, nickel, niobium, molybdenum, tantalum and tungsten, and at least one second element selected from phosphorus, boron, silicon, aluminum and zirconium; and/or
(ii) immobilized Lewis and/or Brønsted acids; and/or
(iii) aluminosilicates.

Suitable multielement oxides are, for example, those comprising 18 to 35% by weight of phosphorus; 11 to 39% by weight of titanium, the molar ratio of phosphorus to titanium being at least 1:1, as described in WO 2013/028356.

Additionally suitable are multielement oxides comprising a mixed oxide of vanadium, titanium, phosphorus and an alkali metal, as described in US 2013/0072716.

Preferably, the multielement oxide is a vanadium-phosphorus oxide. In suitable embodiments, the vanadium-phosphorus oxide has a phosphorus/vanadium atomic ratio of 0.9 to 2.0, preferably of 0.9 to 1.5, more preferably of 0.9 to 1.3 and most preferably of 1.0 to 1.2. The vanadium-phosphorus oxide may be doped with elements other than vanadium and phosphorus.

Preferably, the vanadium-phosphorus oxide corresponds to the general formula (I)

V₁P_{b}X¹_{d}X²ₑOₙ (I)

in which
X¹ is Mo, Bi, Fe, Co, Ni, Si, Zn, Hf, Zr, Ti, Cr, Mn, Cu, B, Sn, Nb and/or Ta, preferably Fe, Nb, Mo, Zn and/or Hf,
X² is Li, K, Na, Rb, Cs and/or TI,
b is 0.9 to 2.0, preferably 0.9 to 1.5, more preferably 0.9 to 1.3 and most preferably 1.0 to 1.2,
d is ≧0 to 0.1,
e is ≧0 to 0.1, and
n is the stoichiometric coefficient of the element oxygen, which is determined by the stoichiometric coefficients of the elements other than oxygen and the valency thereof in (I).

Catalysts comprising an active composition selected from vanadium-phosphorus oxides have been previously described in the literature and are recommended therein especially as catalysts for the heterogeneously catalyzed partial gas phase oxidation of hydrocarbons having at least four carbon atoms (especially n-butane, n-butenes and/or benzene) to maleic anhydride. These catalysts known from the prior art for the aforementioned partial oxidations are suitable as catalysts in the process according to the invention. They feature particularly high selectivities of target product formation (of acrylic acid formation) (with simultaneously high formaldehyde conversions).

Accordingly, the catalysts used in the process according to the invention may, for example, be all of those disclosed in documents U.S. Pat. No. 5,275,996, U.S. Pat. No. 5,641,722, U.S. Pat. No. 5,137,860, U.S. Pat. No. 5,095,125, DE-69702728 T2, WO 2007/012620, WO 2010/072721, WO 2001/68245, U.S. Pat. No. 4,933,312, WO 2003/078310, Journal of Catalysis 107, page 201-208 (1987), DE-A 102008040094, WO 97/12674, “Neuartige Vanadium (IV)-phosphate für die Partialoxidation von kurzkettigen Kohlenwasserstoffen-Synthesen, Kristallstrukturen, Redox-Verhalten and katalytische Eigenschaften [Novel vanadium(IV) phosphates for the partial oxidation of short-chain hydrocarbon syntheses, crystal structures, redox behavior and catalytic properties], thesis by Dipl. Chem. Ernst Benser, 2007, Rheinische Friedrichs-Wilhelms-Universität Bonn”, WO 2010/072723, “Untersuchung von V-P-O-Katalysatoren für die partielle Oxidation von Propan zu Acrylsäure [Study of V-P-O catalysts for the partial oxidation of propane to acrylic acid], thesis by Dipl. Chem. Thomas Quandt, 1999, Ruhr-Universität Bochum”, WO 2010/000720, WO 2008/152079, WO 2008/087116, DE-A 102008040093, DE-A 102005035978 and DE-A 102007005602, and the prior art acknowledged in these documents. In particular, this applies to all exemplary embodiments of the above prior art, especially those of WO 2007/012620.

The mean oxidation state of vanadium in the undoped or doped vanadium-phosphorus oxides is +3.9 to +5.0. In addition, these active compositions advantageously have a specific BET surface area of at least 15 m²/g, preferably of 15 to 50 m²/g and most preferably of 15 to 40 m²/g. It should be emphasized here that all figures in this document for specific surface areas are based on determinations to DIN 66131 (determinations of the specific surface area of solids by gas adsorption (N₂) according to Brunauer-Emmett-Teller (BET)). They advantageously have a total pore volume of at least 0.1 ml/g, preferably of 0.15 to 0.5 ml/g and most preferably of 0.15 to 0.4 ml/g. Figures for total pore volumes in this document are based on measurements by the method of mercury porosymmetry using the Auto Pore 9220 measuring instrument from Micromeritics GmbH, DE-4040 Neuss (range 30 Å to 0.3 mm). As already stated, the active compositions may be doped with promoter elements other than vanadium and phosphorus. Useful promoter elements of this kind include the elements of groups 1 to 15 of the periodic table other than P and V. Doped vanadium-phosphorus oxides are disclosed, for example, by WO 97/12674, WO 95/26817, U.S. Pat. No. 5,137,860, U.S. Pat. No. 5,296,436, U.S. Pat. No. 5,158,923, U.S. Pat. No. 4,795,818 and WO 2007/012620.

Promoters preferred in accordance with the invention are the elements lithium, potassium, sodium, rubidium, cesium, thallium, molybdenum, zinc, hafnium, zirconium, titanium, chromium, manganese, nickel, copper, iron, boron, silicon, tin, cobalt and bismuth, among which preference is given not only to iron but especially to molybdenum, zinc and bismuth. The vanadium-phosphorus oxide active compositions may comprise one or more promoter elements. The total content of promoters in the active composition is, based on the weight thereof, generally not more than 5% by weight (in each case calculating the individual promoter elements as the electrically uncharged oxide in which the promoter element has the same valency (oxidation number) as in the active composition).

The catalyst may comprise the multielement oxide, preferably the vanadium-phosphorus oxide, for example, in pure undiluted form or diluted with an oxidic, essentially inert diluent material as what is called an unsupported catalyst. Suitable inert diluent materials include, for example, finely divided alumina, silica, aluminosilicates, zirconia, titania or mixtures thereof. Undiluted unsupported catalysts are preferred in accordance with the invention. The unsupported catalysts may in principle be in any shape. Preferred unsupported catalysts are spheres, solid cylinders, hollow cylinders and trilobes, the longest dimension of which in all cases is advantageously 1 to 10 mm.

In the case of shaped unsupported catalyst bodies, the shaping is advantageously effected with precursor powder, which is calcined only after the shaping. The shaping is typically effected with addition of shaping aids, for example graphite (lubricant) or mineral fibers (reinforcing aid). Suitable shaping processes include tabletting and extrusion.

Appropriately in application terms, the external diameter of cylindrical unsupported catalysts is 3 to 10 mm, preferably 4 to 8 mm and in particular 5 to 7 mm. The height thereof is advantageously 1 to 10 mm, preferably 2 to 6 mm and in particular 3 to 5 mm. The same applies in the case of hollow cylinders. In addition, the internal diameter of the orifice which passes through from the top downward is advantageously 1 to 8 mm, preferably 2 to 6 mm and most preferably 2 to 4 mm. A wall thickness of 1 to 3 mm is appropriate in application terms in the case of hollow cylinders.

The doped or undoped active composition may also be used in powder form or in the form of eggshell catalysts with an active composition shell applied to the surface of inert shaped support bodies as the catalyst. The production of the eggshell catalysts involves using a pulverulent active composition or using a pulverulent, as yet uncalcined precursor composition with additional use of a liquid binder to coat the surface of an inert shaped support body (if coating is effected with uncalcined precursor composition, the calcination follows the coating and generally drying). Inert shaped support bodies normally also differ from the active composition in that they have a much lower specific surface area. In general, the specific surface area thereof is less than 3 m²/g of shaped support bodies.

Suitable materials for aforementioned inert shaped support bodies are, for example, quartz, silica glass, sintered silica, sintered or fused alumina, porcelain, sintered or fused silicates such as aluminum silicate, magnesium silicate, zinc silicate, zirconium silicate and especially steatite (e.g. C 220 steatite from CeramTec). The geometry of the inert shaped support bodies may in principle be irregular, preference being given in accordance with the invention to shaped support bodies of regular shape, for example spheres or hollow cylinders. Appropriately in application terms, the longest dimension of the aforementioned inert shaped support bodies for the inventive purposes is 1 to 10 mm.

The coating of the inert shaped support bodies with the respective fine powder is generally executed in a suitable rotatable vessel, for example in a coating drum. Appropriately in application terms, the liquid binder is sprayed onto the inert shaped support bodies and the binder-moistened surface of the shaped support bodies agitated in the coating drum is dusted with the particular powder (cf., for example, EP-A 714 700). Subsequently, the adhesion liquid is generally removed at least partly from the coated shaped support body (for example by passing hot gas through the coated shaped support bodies, as described by WO 2006/094766). In principle, however, it is also possible to employ all other application processes acknowledged as prior art in EP-A 714 700 for production of the relevant eggshell catalysts. Useful liquid binders include, for example, water and aqueous solutions (e.g. of glycerol in water). For example, the coating of the shaped support bodies can also be undertaken by spraying a suspension of the pulverulent composition to be applied in liquid binder (e.g. water) onto the surface of the inert shaped support bodies (generally under the action of heat and a drying entraining gas). In principle, the coating can also be undertaken in a fluidized bed system or powder coating system.

The layer thickness of the active composition applied to the surface of the inert shaped support body is, appropriately in application terms, selected within the range from 10 to 2000 μm, or 10 to 500 μm, or 100 to 500 μm, or 200 to 300 μm. Suitable eggshell catalysts include those whose inert shaped support body is a hollow cylinder having a length of 3 to 6 mm, an external diameter in the range from 4 to 8 mm and a wall thickness in the range from 1 to 2 mm. In addition, all ring geometries disclosed in DE-A 102010028328 and in DE-A 102010023312 and in EP-A 714 700 for possible inert shaped support bodies of annular catalysts are suitable.

Preference is given to obtaining shaped unsupported catalyst bodies whose active composition is a vanadium-phosphorus oxide by reacting a pentavalent vanadium compound, preferably V₂O₅, with an organic reducing solvent, preferably isobutanol, in the presence of a pentavalent phosphorus compound, preferably ortho- and/or pyrophosphoric acid, to give a catalyst precursor composition, shaping the catalyst precursor composition to shaped catalyst precursor bodies and calcining (thermally treating) them at a temperature in the range from 200 to 500° C.

For example, the production of the shaped unsupported catalyst bodies may comprise the following steps:
a) reacting a pentavalent vanadium compound (e.g. V₂O₅) with an organic reducing solvent (e.g. alcohol, for instance isobutanol) in the presence of a pentavalent phosphorus compound (e.g. ortho- and/or pyrophosphoric acid) with heating to 75 to 205° C., preferably to 100 to 120° C.;
b) cooling the reaction mixture to advantageously 40 to 90° C.;
c) isolating the solid V, P, O-comprising precursor composition formed (for example by filtering);
d) drying and/or thermally pretreating the precursor composition (optionally until commencement of preforming by elimination of water from the precursor composition);
e) adding shaping aids, for example finely divided graphite or mineral fibers, and then shaping to give the shaped catalyst precursor body, for example by tabletting;
f) subsequently at least once calcining the shaped catalyst precursor bodies formed by heating in an atmosphere comprising oxygen, nitrogen, noble gases, carbon dioxide, carbon monoxide and/or steam. In the course of calcination, the temperature generally exceeds 250° C., preferably 300° C., more preferably 350° C., but does not normally exceed 700° C., preferably 650° C. and most preferably 600° C.

Preference is given to a calcination in which the catalyst precursor
(i) is heated in at least one calcination zone in an oxidizing atmosphere having an oxygen content of 2 to 21% by volume to a temperature of 200 to 350° C. and leaving it under these conditions until the desired mean oxidation state of the vanadium is attained; and
(ii) is heated in at least one further calcination zone in a nonoxidizing atmosphere having an oxygen content of ≦0.5% by volume and a hydrogen oxide content of 20 to 75% by volume to a temperature of 300 to 500° C. and leaving it under these conditions for ≧0.5 hour.

The catalyst precursor may either be a shaped catalyst precursor body or a precursor composition.

In step (i), the catalyst precursor is left in an oxidizing atmosphere having a content of molecular oxygen of generally 2 to 21% by volume and preferably of 5 to 21% by volume at a temperature of 200 to 350° C. and preferably of 250 to 350° C. over a period which is effective for establishing the desired mean oxidation state of the vanadium. In general, in step (i), mixtures of oxygen, inert gases (e.g. nitrogen or argon), hydrogen oxide (steam) and/or air and air are used. From the point of view of the catalyst precursor conducted through the calcination zone(s), the temperature during the calcination step (i) can be kept constant, or rise or fall on average. Since step (i) is generally preceded by a heating phase, the temperature will generally first rise, and then even out at the desired final value. In general, therefore, the calcination zone of step (i) is preceded upstream by at least one further calcination zone for heating of the catalyst precursor.

The period over which the heat treatment in step (i) is maintained in the process according to the invention should be selected such that a mean oxidation state of the vanadium is adjusted to a value of +3.9 to +5.0.

Since the determination of the mean oxidation state of the vanadium during the calcination can be determined only with extreme difficulty for reasons of apparatus and time, the period required should advantageously be determined experimentally in preliminary tests. In general, this is accomplished by a test series in which heat treatment is effected under defined conditions, the samples being removed from the system, cooled and analyzed for the mean oxidation state of the vanadium after different times.

The period required in step (i) is generally dependent on the nature of the catalyst precursor, the temperature set and the gas atmosphere selected, especially the oxygen content. In general, the period in step (i) extends to a duration of more than 0.5 hour and preferably of more than 1 hour. In general, a period of up to 4 hours, preferably of up to 2 hours, is sufficient to establish the desired mean oxidation state. Under appropriate conditions (for example lower region of the temperature interval and/or low content of molecular oxygen), however, a period of more than 6 hours may also be required.

In step (ii), the catalyst intermediate obtained is left in a nonoxidizing atmosphere having a content of molecular oxygen of ≦0.5% by volume and of hydrogen oxide (steam) of 20 to 75% by volume, preferably of 30 to 60% by volume, at a temperature of 300 to 500° C. and preferably of 350 to 450° C. over a period of 0.5 hour, preferably 2 to 10 hours and more preferably 2 to 4 hours. The nonoxidizing atmosphere comprises, as well as the hydrogen oxide mentioned, generally predominantly nitrogen and/or noble gases, for example argon, though this should not be understood as a restriction. Gases such as carbon dioxide, for example, are also suitable in principle.

The nonoxidizing atmosphere preferably comprises ≧40% by volume of nitrogen. From the point of view of the catalyst intermediate conducted through the calcination zone(s), the temperature during the calcination step (ii) can be kept constant, or rise or fall on average. If step (ii) is performed at a higher or lower temperature than step (i), there is generally a heating or cooling phase between steps (i) and (ii), which is optionally implemented in a further calcination zone. In order to enable an improved separation from the oxygenous atmosphere of step (i), this further calcination zone may be purged between (i) and (ii), for example for purging with inert gas, for example nitrogen. Preference is given to performing step (ii) at a temperature 50 to 150° C. higher than step (i).

In general, the calcination comprises a further step (iii) to be performed after step (ii), in which the calcined catalyst precursor is cooled in an inert gas atmosphere to a temperature of ≦300° C., preferably of ≦200° C. and more preferably of ≦150° C.

Before, between and/or after steps (i) and (ii), or (i), (ii) and (iii), further steps are possible in the calcination in the process according to the invention. Without any limiting effect, examples of further steps include changes in the temperature (heating, cooling), changes in the gas atmosphere (adjustment of the gas atmosphere), further hold times, transfers of the catalyst intermediates to other apparatuses or interruptions in the overall calcination process.

Since the catalyst precursor is generally at a temperature of <100° C. before commencement of the calcination, it typically has to be heated before step (i). The heating can be performed with employment of various gas atmospheres. Preference is given to performing the heating in an oxidizing atmosphere, as defined in step (i), or an inert gas atmosphere, as defined in step (iii). An exchange of the gas atmosphere during the heating phase is also possible. Particular preference is given to heating in the oxidizing atmosphere which is also employed in step (i).

Other suitable condensation catalysts are selected from immobilized Lewis and/or Brønsted acids. The Lewis and/or Brønsted acids are preferably immobilized on solid supports, especially solid porous supports. Suitable Lewis acids are, for example, oxides of tungsten, niobium or lanthanum, or mixtures of two or more of these oxides, such as WO₃, Nb₂O₅, NbOPO₄ and La₂O₃. These oxides can be prepared in a manner which is customary per se, for example by calcination in oxygenous atmosphere of, for example, ammonium tungstate ((NH₄)₂WO₄) or ammonium niobate (NH₄NbO₃).

Suitable supports are, for example, TiO₂, SiO₂, Al₂O₃ and carbon supports.

The supports serve predominantly to increase the specific surface area or to fix the active sites. The supported catalysts can be produced in various ways by methods which are conventional per se, for example by saturating or impregnating the support material, for example by means of the incipient wetness method, by spraying with a solution of a precursor compound, preferably an aqueous solution, and then drying and calcining the solids thus obtained to give the catalysts usable in accordance with the invention.

Preferably, the immobilized Lewis and/or Brønsted acid is selected from immobilized heteropolyacids. Heteropolyacids comprise polyoxo anions having a negative charge (e.g. [PW₁₂O₄₀]³⁻) balanced by cations, including at least one proton. Polyoxo anions are cage structures usually including one or more generally central atoms surrounded by a cage structure. The cage structure has a plurality of oxygen-bonded metal atoms which may be the same or different. The central atom is (the central atoms are) different than the atoms of the cage base structure. Most of the heteropolyacids and polyoxometallates have a tetrahedrally bonded central atom (X) bonded via four oxygen atoms to the metal atoms (M¹). The metal atoms in turn are normally bonded octahedrally via oxygen atoms (O) to the central atom, and bonded to four other metal atoms via oxygen atoms. The metal atoms also have a sixth, non-bridging oxygen atom, which is also referred to as terminal oxygen. In general, the metal atom is selected from molybdenum, tungsten, vanadium, chromium, niobium, tantalum and titanium.

Heteropolyacids occur in the form of various known structures, such as the Keggin, Dawson and Anderson structures.

The heteropolyacid preferably corresponds to the formula (II)

H_{(f-a*z)}Zₐ[X_{b}M¹_{c}M²_{d}Oₑ] (II)

in which
Z is a cation other than H⁺,
a is a number from 1 to 30,
z is the charge of the cation Z,
f is the charge of the anion [X_{b}M¹_{c}M²_{d}Oₑ]^{f-},
(f−a*z) is greater than 0,
X is at least one element selected from phosphorus, silicon, germanium, antimony, boron, arsenic, aluminum, tellurium and cerium,
b is a number from 1 to 5,
M¹ is at least one metal selected from chromium, molybdenum, vanadium, tungsten, niobium, tantalum and titanium,
c is a number from 3 to 20, preferably 5 to 20,
M² is at least one metal selected from the metals of groups 3 to 10 of the periodic table of the elements and zinc, but is not chromium, molybdenum, vanadium, tungsten, niobium, tantalum or titanium,
d is a number in the range from 0 to 6, preferably 1 to 6, and
e is the stoichiometric coefficient of the element oxygen, which is determined by the stoichiometric coefficients of the elements other than oxygen and the valency thereof in (II).

In formula (II) “a*z” is the product obtained by multiplying “a” by “z”.

Preferably, M² is at least one metal selected from the metals iron, cobalt, nickel, ruthenium, rhodium, palladium, osmium, iridium, platinum and manganese.

In a typical Keggin heteropolyacid, b is 1, c is 12 and e is 40, as in H₃PMo₁₂O₄₀. In a typical Dawson heteropolyacid, b is 2, c is 18 and e is 62, as in H₆P₂Mo₁₈O₆₂.

Heteropolyacids are commercially available or can be produced by a multitude of known methods. Syntheses of heteropolyacids are described in general terms in Pope et. al., Heteropoly and Isopoly Oxometallates, Springer-Verlag, New York (1983). Typically, heteropolyacids are prepared by mixing the desired metal oxides with water, adjusting the pH to about 1 to 2 for provision of the required protons with acid, for example hydrochloric acid, and then vaporizing water until the desired heteropolyacid precipitates out. For example, the heteropolyacid H₃PMo₁₂O₄₀ can be prepared by combining Na₂HPO₄ and Na₂MoO₄, adjusting the pH with sulfuric acid, extracting with ether and crystallizing the resulting heteropolyacid in water. Vanadium-substituted heteropolyacids can be prepared by the method described in V. F. Odyakov, et al., Kinetics and Catalysis, 1995, vol. 36, p. 733.

The heteropolyacid is immobilized by application to a support. The support materials used may, for example, be alumina, titania, silica, zirconia, carbon supports or mixtures thereof.

In condensation catalysts selected from aluminosilicates, the aluminosilicate is preferably a zeolite. The zeolite is preferably selected from zeolites of the MFI and MOR types. Preferably, the zeolite has a silicon/aluminum atomic ratio of more than 10. More preferably, the zeolite is selected from the zeolites of the MFI and MOR types and has a silicon/aluminum atomic ratio of more than 10.

Before the catalyst is contacted with the reaction gas, a so-called activation can be performed in the reactor. The activation involves passing an activating gas mixture comprising molecular oxygen at a temperature of 200 to 450° C. over the catalyst. The activation may extend over a few minutes up to a few days. Preferably, the pressure of the activating gas mixture and the residence time thereof over the catalyst in the course of activation are set similarly to the pressure of the reaction gas and residence time thereof over the catalyst in the course of preparation of acrylic acid. The activating gas mixture comprises molecular oxygen and at least one inert activating gas constituent selected from N₂, CO, CO₂, H₂O and noble gases such as Ar. In general, the activating gas comprises 0.5 to 22% by volume, preferably 1 to 20% by volume and especially 1.5 to 18% by volume of molecular oxygen. Preference is given to using air as a constituent of the activating gas mixture.

The residence time of the reaction gas in contact with the catalyst is not restricted. It is generally in the range of 0.3-15.0 s, preferably 0.7-13.5 s and more preferably 1.0-12.5 s. The ratio of flow of reaction gas based on the volume of the catalyst is 200-5000 h⁻¹, preferably 250-4000 h⁻¹ and even more preferably 300-3500 h⁻¹.

The load of formaldehyde source on the catalyst, calculated as formaldehyde equivalent (expressed in g_{formaldehyde}/(g_{catalyst}*hour)), is generally 0.01-3.0 h⁻¹, preferably 0.015-1.0 h⁻¹ and even more preferably 0.02-0.5 h⁻¹. “g_{catalyst}*hour” is the product obtained by multiplying “g_{catalyst}” and “hour”.

Preferably, the formaldehyde source is selected from formaldehyde, trioxane, paraformaldehyde, formalin, methylal, aqueous paraformaldehyde solution and aqueous formaldehyde solution, or is provided by heterogeneously catalyzed partial gas phase oxidation of methanol.

Trioxane is a heterocyclic compound which forms through trimerization of formaldehyde and decomposes in the course of heating to give monomeric formaldehyde. Since the reaction gas is contacted with the catalyst at elevated temperature (generally more than 250° C.), trioxane is a formaldehyde source of good suitability. Since trioxane dissolves in water and in alcohols such as methanol, corresponding trioxane solutions can also be used as the formaldehyde source for the process according to the invention. A sulfuric acid content in trioxane solutions of 0.25 to 0.50% by weight promotes the splitting to formaldehyde. Alternatively, the trioxane can also be dissolved in a liquid consisting principally of acetic acid, and the resulting solution can be vaporized for the purposes of generating the reaction gas and the trioxane present therein can be split into formaldehyde at the elevated temperature.

Aqueous formaldehyde solution can be purchased commercially, for example, with a formaldehyde content of 35 to 50% by weight as formalin. Typically, formalin comprises small amounts of methanol as a stabilizer. These may, based on the weight of the formalin, be 0.5 to 20% by weight, preferably 0.5 to 5% by weight and more preferably 0.5 to 2% by weight. After conversion to the vapor phase, the formalin can be used directly for provision of the reaction gas.

In the process described here, it is possible in principle, inter alia, to use all aqueous formaldehyde solutions at 1-100% by weight. Preference is given, however, to concentrated formaldehyde solutions as the feedstock between 48-90% by weight, or more preferably 60-80% by weight, of formaldehyde in aqueous solution. Corresponding processes for concentrating such formaldehyde solutions are prior art and are described, for example, in WO 04/078690, WO 04/078691 or WO 05/077877.

Paraformaldehyde is the short-chain polymer of formaldehyde, the degree of polymerization of which is typically 8 to 100. This is a white powder which is split into formaldehyde at low pH values or while heating.

In the course of heating of paraformaldehyde in water, it decomposes, giving an aqueous formaldehyde solution likewise suitable as a formaldehyde source. Sometimes, it is referred to as aqueous “paraformaldehyde solution” in order to delimit it for terminology purposes from aqueous formaldehyde solutions which are obtained by dilution of formalin. In fact, paraformaldehyde as such, however, is essentially insoluble in water.

Methylal (dimethoxymethane) is the reaction product of formaldehyde with methanol, which is in the form of a colorless liquid at standard pressure and 25° C. It is cleaved hydrolytically in aqueous acids to form formaldehyde and methanol. After conversion to the vapor phase, both methylal and the hydrolyzate formed in aqueous acid can be used directly for the provision of the reaction gas.

On the industrial scale, formaldehyde is prepared by heterogeneously catalyzed partial gas phase oxidation of methanol. It is particularly preferable in accordance with the invention to provide the formaldehyde by heterogeneously catalyzed partial gas phase oxidation of methanol. The formaldehyde is supplied to the reaction gas in this embodiment as the product gas of a heterogeneously catalyzed partial gas phase oxidation of methanol to formaldehyde, optionally after some or all of the methanol and/or molecular oxygen present in the product gas has been removed.

In one embodiment, the reaction gas comprises only a small proportion of molecular oxygen, if any. In this embodiment, the ratio of the partial pressure of the molecular oxygen in the reaction gas to the total pressure of the reaction gas is preferably less than 0.015, more preferably less than 0.01 and most preferably 0 to 0.005.

In an alternative embodiment, the reaction gas comprises molecular oxygen. In this embodiment, the ratio of the partial pressure of the molecular oxygen in the reaction gas to the total pressure of the reaction gas is, for example, 0.018 to 0.1, or 0.02 to 0.05, preferably 0.02 to 0.04.

In order to increase the activity of the catalyst, a regeneration step can be performed between every two production steps in which the acrylic acid is prepared. In the regeneration step, a regeneration gas mixture comprising molecular oxygen is passed over the catalyst at a temperature of 200 to 450° C. The regeneration step may extend over a few minutes up to a few days. Preferably, the pressure of the regeneration gas mixture and the residence time thereof over the catalyst in the regeneration step are set similarly to the pressure of the reaction gas and residence time thereof over the catalyst in the production step. The regeneration gas mixture comprises molecular oxygen and at least one inert regeneration gas constituent selected from N₂, CO, CO₂, H₂O and noble gases such as Ar. In general, the oxygenous regeneration gas comprises 0.5 to 22% by volume, preferably 1 to 20% by volume and especially 1.5 to 18% by volume of molecular oxygen. Preference is given to using air as a constituent of the regeneration gas mixture.

In a preferred embodiment of the process, the acrylic acid is obtained by fractional condensation of the product gas. This involves reducing the temperature of the product gas, optionally at first by direct and/or indirect cooling, and then passing it into a condensation zone within which the product gas fractionally condenses while ascending into itself. The condensation zone is preferably within a condensation column which is equipped with separating internals (for example mass transfer trays) and optionally provided with cooling circuits. Through appropriate selection of the number of theoretical plates, the acrylic acid is obtained in the form of a first fraction consisting predominantly, preferably at least to an extent of 90% by weight, more preferably at least to an extent of 95% by weight, of acrylic acid. It is particularly preferable to configure the fractional condensation, more particularly in terms of the number of theoretical plates, such that, as well as the acrylic acid in the form of the first fraction, the unconverted acetic acid is obtained in the form of a second fraction consisting predominantly, preferably at least to an extent of 90% by weight, more preferably at least to an extent of 95% by weight, of acetic acid.

In an alternative preferred embodiment of the process, the acrylic acid is obtained by absorption into an absorbent and subsequent rectification of the laden absorbent out of the product gas. This involves reducing the temperature of the product gas by direct and/or indirect cooling and contacting it in an absorption zone with an organic absorbent having a higher boiling point than acrylic acid at standard pressure. Useful organic absorbents include, for example, those mentioned in DE-A 102009027401 and in DE-A 10336386. As well as the acrylic acid, acetic acid is generally also absorbed into the absorbent. Preferably, the absorption zone is within an absorption column preferably equipped with separating internals. The acrylic acid is obtained from the laden absorbent by rectification. In the course of rectification, through appropriate selection of the number of theoretical plates, the acrylic acid is obtained in the form of a first fraction consisting predominantly, preferably at least to an extent of 90% by weight, more preferably at least to an extent of 95% by weight, of acrylic acid. It is particularly preferable to configure the fractional condensation, especially in terms of the number of theoretical plates, such that, as well as the acrylic acid in the form of the first fraction, the unconverted acetic acid is obtained in the form of a second fraction consisting predominantly, preferably at least to an extent of 90% by weight, more preferably at least to an extent of 95% by weight, of acetic acid.

The molar ratio of the acetic acid to the formaldehyde source, calculated as formaldehyde equivalents, in the process according to the invention is preferably 2 to 10, more preferably 2 to 6 and most preferably 2.5 to 5.

The greater the molar ratio of the acetic acid to the formaldehyde source, calculated as formaldehyde equivalents, the greater the amount of acetic acid which is not converted over the catalyst and is consequently present in the product gas. The loss of unconverted acetic acid which occurs via the product gas can thus be considerable if only the acrylic acid prepared in accordance with the invention is obtained from the product gas and utilized. In order to keep the loss of acetic acid as low as possible, in a preferred embodiment of the process, at least a portion of the acetic acid present in the product gas is recycled. “Recycling” is understood to mean that at least a portion of the acetic acid present in the product gas is used as at least a portion of the acetic acid encompassed by the reaction gas. Preference is given to recycling the acetic acid in the form of the second fraction of the fractional condensation or of the rectification, which, as described above, consists predominantly of the acetic acid.

In one embodiment, the process according to the invention comprises the preparation of the acetic acid by partial oxidation of ethanol, wherein a gas mixture comprising ethanol and molecular oxygen is converted in contact with at least one solid oxidation catalyst, the active composition of which is preferably a vanadium oxide, to give a product gas mixture. This involves oxidizing ethanol with molecular oxygen under heterogeneous catalysis to give acetic acid and steam. The conditions, especially temperature and pressure, are adjusted such that ethanol, acetic acid and water are present in gaseous form or very predominantly in gaseous form. The product gas mixture can be used directly as part of the inventive reaction gas.

In an alternative embodiment, the process according to the invention comprises the preparation of the acetic acid by homogeneously catalyzed carbonylation of methanol, wherein methanol and carbon monoxide are converted in the liquid phase at a pressure of at least 30 bar (absolute). The conversion is effected in the presence of a catalyst comprising at least one of the elements Fe, Co, Ni, Ru, Rh, Pd, Cu, Os, Ir and Pt, an ionic halide and/or a covalent halide, and optionally a ligand, for example PR³ or NR³, where R is an organic radical.

### EXAMPLES

### Preparation of the Condensation Catalyst

A nitrogen-inertized 8 m³ steel/enamel stirred tank which is externally heatable by means of pressurized water and has baffles was initially charged with 4602 kg of isobutanol. After switching on the three-stage impeller stirrer, the isobutanol was heated to 90° C. under reflux. At this temperature, the addition of 690 kg of vanadium pentoxide via a conveying screw was then commenced. After about ⅔ of the desired amount of vanadium pentoxide had been added after about 20 minutes, the pumped introduction of 805 kg of 105% phosphoric acid was commenced with further addition of vanadium pentoxide. After addition of the phosphoric acid, the reaction mixture was heated under reflux to about 100 to 108° C. and left under these conditions for 14 hours. Subsequently, the mixture was discharged into a previously nitrogen-inertized and heated pressure suction filter and filtered at a temperature of about 100° C. at a pressure above the suction filter of up to 3.5 bar. The filtercake was dried by constantly introducing nitrogen at 100° C. and while stirring with a centered, height-adjustable stirrer within about one hour. The dried filtercake was heated to about 155° C. and evacuated to a pressure of 150 mbar. The drying was performed down to a residual isobutanol content of <2% by weight in the dried catalyst precursor. The dried powder obtained was then heat treated under air in a rotary tube having a length of 6.5 m, an internal diameter of 0.9 m and internal spirals for 2 hours. The speed of the rotary tube was 0.4 rpm. The powder was conveyed into the rotary tube in a volume of 60 kg/h. The air feed was 100 m³/h. The temperatures of the five heating zones of equal length, measured on the outside of the rotary tube, were 250° C., 300° C., 340° C., 340° C. and 340° C. After cooling to room temperature, the heat-treated catalyst precursor was mixed intimately with 1% by weight of graphite and compacted in a roll compactor. The fines in the compactate having a particle size of <400 μm were sieved off and fed back to the compacting operation. The coarse material having a particle size of >400 μm was mixed intimately with a further 2% by weight of graphite. The heat-treated catalyst precursor was compressed in a tableting machine to give hollow cylindrical shaped catalyst precursor bodies having dimensions of 5.5×3.2×3 mm (diameter x height x diameter of the inner hole). The compression forces were about 10 kN.

About 2.7 t of the shaped catalyst precursor bodies were added continuously in a bed height of 9 to 10 cm to the gas-permeable conveyor belt of a belt calcining apparatus composed of two successive, identical belt calcining apparatuses having a total of eight calcining zones. The first 1.4 t were used for single adjustment of the operating parameters of the belt calcining apparatus. Since they were not a homogeneous material, they were not used any further subsequently. The belt calcining apparatus was operated at atmospheric pressure. Between calcining zones 4 and 5 was an encapsulated transition zone. Each of the eight calcining zones comprised a ventilator for generation of gas circulation. Each of the eight calcining zones was supplied with the desired amount of desired fresh gas. To maintain the desired atmospheric pressure, an appropriate amount of gas was removed. The volume of the gas circulating in each calcination zone per unit time was greater than the volume of the gas supplied or removed per unit time. Between every two successive calcination zones was a dividing wall for reduction of gas exchange, which was open in the region of the flow of the catalyst precursor. The length of each calcining zone was 1.45 m. The speed of the conveyor belt was adjusted according to the desired residence time of about 2 hours per calcining zone. The individual zones were operated as shown in the following table:

| Calcination | | | | | Transition | | | | |
|---|---|---|---|---|---|---|---|---|---|
| zone No. | 1 | 2 | 3 | 4 | zone | 5 | 6 | 7 | 8 |
| Temperature | 140 | 140 | 260 | 300 | Cooling to | 335 | 400 | 425 | 355 |
| [° C.] | | | | | 200° C. | | | | |
| fresh gas | air | air | air | air | air | N₂ | N₂/H₂O | N₂/H₂O | N₂ |
| supplied | | | | | | | (1:1) | (1:1) | |

### Experimental Plant:

An experimental plant equipped with a feed metering unit and an electrically heated vertical reactor tube was used. The reactor used (stainless steel materials No. 1.4541) had a tube length of 950 mm, an external diameter of 20 mm and an internal diameter of 16 mm. Around the reactor were mounted four copper half-shells (E-Cu F25, external diameter 80 mm, internal diameter 16 mm, length 450 mm). A heating band was wound around the half-shells, and insulating tape was in turn wound around this. The temperature of the reactor heaters was measured on the outside of the heating shell of the reactor. In addition, it was possible to determine the temperature within the reactor over the entire catalyst bed with the aid of a thermocouple present in a central sleeve (external diameter 3.17 mm, internal diameter 2.17 mm). At the lower end of the reactor tube, a wire mesh of a so-called catalyst seat prevented the discharge of the catalyst bed. The catalyst seat consisted of a tube of length 5 cm (external diameter 14 cm, internal diameter 10 cm), over the upper orifice of which the wire mesh (mesh size 1.5 mm) was present. In the reactor tube, 14 g of a further bed composed of steatite spheres having a diameter of 3-4 mm (bed height 5 cm) was placed upon this catalyst seat. The thermocouple sleeve was placed centrally onto the further bed. Then 105 g in each case of catalyst in the form of spall of grain size 2.0 to 3.0 mm were introduced undiluted around the thermocouple sleeve into the reaction tube (bed height 66 cm). Above the catalyst bed were 14 g of a preliminary bed composed of steatite spheres having a diameter of 3-4 mm (bed height 5 cm).

### Operation of the Experimental Plant:

A solution of trioxane in acetic acid was initially charged in a reservoir vessel under a nitrogen atmosphere. The molar ratio of trioxane, calculated as formaldehyde (Fa), to acetic acid (HOAc) was as specified in table 1. A Desaga KP 2000 pump was used to meter in the desired volume flow rate of the solution, and it was conveyed into a vaporizer coil. The solution was vaporized at 85° C. in the presence of preheated nitrogen. The gas mixture was heated to 180° C. in a preheater and conducted through the reactor heated to 310° C. The pressure of the reaction gas was adjusted manually to 1.15 bar+/−0.05 bar. All gas flow rates were monitored by means of mass flow meters. Analysis stubs at the reactor inlet and outlet enabled the analysis of the gas composition by online GC analysis.

The compositions of the product gas were determined by gas chromatography.

The compositions of the product gas measured after 30 minutes, 4 hours and 10 hours were used to calculate the space-time yield of acrylic acid prepared (STY_{AA}) attained at these times. The space-time yield of acrylic acid prepared is based on the mass of acrylic acid in g which is formed per liter of catalyst per hour. The results are reported in table 1.

TABLE 1

| | | | Reactants | Space-time yield | | |
|---|---|---|---|---|---|---|
| | | p_{Fa}** | [% by vol.] | STY_{AA} [gl⁻¹h⁻¹] | | |
| Ex. | n_{HOAc}:n_{Fa}*** | [mbar] | (HOAc/Fa/N₂) | 30 min | 4 h | 10 h |
| 1* | 3:1 | 52 | 13.7/4.5/81.8 | 31 | 26 | 21 |
| 2 | 3:1 | 105 | 27.4/9.1/63.5 | 60 | 46 | 34 |
| 3 | 3:1 | 158 | 41.0/13.7/45.3 | 84 | 63 | 45 |
| 4* | 4.4:1 | 53 | 20.3/4.6/75.1 | 32 | 29 | 26 |
| 5 | 4.4:1 | 106 | 40.8/9.2/50.0 | 72 | 62 | 44 |
| 6 | 4.4:1 | 159 | 61.2/13.8/25.0 | 108 | 89 | 70 |

*comparative example
**partial formaldehyde pressure
***molar ratio of acetic acid to formaldehyde

1. A process for preparing acrylic acid, the process comprising:
contacting a reaction gas with a solid condensation catalyst, thereby obtaining a product gas comprising the acrylic acid,
wherein
the reaction gas comprises a gaseous formaldehyde source and gaseous acetic acid,
a partial pressure of the formaldehyde source, calculated as formaldehyde equivalents, is at least 85 mbar, and
a molar ratio of the acetic acid to the formaldehyde source, calculated as formaldehyde equivalents, is at least 1.

2. The process according to claim 1, wherein the partial pressure of the formaldehyde source, calculated as formaldehyde equivalents, is at least 100 mbar.

3. The process according to claim 1, wherein a ratio of the partial pressure of the formaldehyde source, calculated as formaldehyde equivalents, to a total pressure of the reaction gas is from 0.1 to 0.5.

4. The process according to claim 1, wherein a ratio of a partial pressure of the acetic acid to a total pressure of the reaction gas is from 0.5 to 0.9.

5. The process according to claim 1, wherein the molar ratio of the acetic acid to the formaldehyde source, calculated as formaldehyde equivalents, is from 2 to 10.

6. The process according to claim 1, wherein the reaction gas comprises an inert diluent gas.

7. The process according to claim 1, wherein said contacting occurs at a reaction temperature of from 250 to 400° C.

8. The process according to claim 1, wherein the condensation catalyst is at least one selected from the group consisting of
(i) a catalyst having an active composition which comprises a multielement oxide and at least one first element selected from the group consisting of titanium, vanadium, chromium, iron, cobalt, nickel, niobium, molybdenum, tantalum and tungsten, and at least one second element selected from the group consisting of phosphorus, boron, silicon, aluminum and zirconium;
(ii) an immobilized Lewis and/or Brønsted acid; and
(iii) an aluminosilicate.

9. The process according to claim 8, wherein the condensation catalyst is the immobilized Lewis and/or Brønsted acid, which is an immobilized heteropolyacid.

10. The process according to claim 8, wherein
the condensation catalyst is the catalyst having an active composition which comprises a multielement oxide and at least one first element selected from the group consisting of titanium, vanadium, chromium, iron, cobalt, nickel, niobium, molybdenum, tantalum and tungsten, and at least one second element selected from the group consisting of phosphorus, boron, silicon, aluminum and zirconiumthe, and
the multielement oxide is a vanadium-phosphorus oxide having a phosphorus/vanadium atomic ratio of from 0.9 to 2.0.

11. The process according to claim 10, wherein the vanadium-phosphorus oxide corresponds to formula (I)
V₁P_{b}X¹_{d}X²ₑOₙ (I)
where
X¹ is Mo, Bi, Fe, Co, Ni, Si, Zn, Hf, Zr, Ti, Cr, Mn, Cu, B, Sn, Nb and/or Ta,
X² is Li, K, Na, Rb, Cs and/or Tl,
b is a number of from 0.9 to 2.0,
d is a number of from 0 to 0.1,
e is a number of from 0 to 0.1, and
n is a stoichiometric coefficient of oxygen, which is determined by stoichiometric coefficients of elements other than oxygen and valency thereof in formula (I).

12. The process according to claim 9, wherein the heteropolyacid corresponds to formula (II)
H_{(f-a*z)}Zₐ[X_{b}M¹_{c}M²_{d}Oₑ] (II)
where
Z is a cation other than H⁺,
a is a number of from 1 to 30,
z represents charge of cation Z,
f represents charge of anion [X_{b}M¹_{c}M²_{d}Oₑ]^{f-},
(f−a*z) is greater than 0,
X is at least one element selected from the group consisting of phosphorus, silicon, germanium, antimony, boron, arsenic, aluminum, tellurium and cerium,
b is a number of from 1 to 5,
M¹ is at least one metal selected from the group consisting of chromium, molybdenum, vanadium, tungsten, niobium, tantalum and titanium,
c is a number of from 3 to 20,
M² is at least one metal selected from the group consisting of a metal of groups 3 to 10 of the periodic table and zinc, excluding chromium, molybdenum, vanadium, tungsten, niobium, tantalum and titanium,
d is a number of from 0 to 6, and
e is a stoichiometric coefficient of oxygen, which is determined by stoichiometric coefficients of elements other than oxygen and valency thereof in formula (II).

13. The process according to claim 8, wherein the condensation catalyst is the aluminosilicate, which is a zeolite.

14. The process according to claim 1, wherein the acrylic acid is obtained by fractional condensation of the product gas.

15. The process according to claim 1, wherein the acrylic acid is obtained from the product gas by absorption into an absorbent to obtain a laden absorbent and subsequent rectification of the laden absorbent from the product gas.

16. The process according to claim 1, wherein the formaldehyde source
is selected from the group consisting of formaldehyde, trioxane, paraformaldehyde, formalin, methylal, an aqueous paraformaldehyde solution and an aqueous formaldehyde solution, or
is provided by heterogeneously catalyzed partial gas phase oxidation of methanol.
